(19) United States
(12) Patent Application Publication
(10) Pub. No.: US 2013/0103153 A1
(43) Pub. Date: Apr. 25, 2013

(54) INTERBODY IMPLANT SYSTEM AND METHODS OF USE

(71) Applicants: Jonathan Blackwell, Arlington, TN (US); Thomas E. Drochner, Memphis, TN (US)

(72) Inventors: Jonathan Blackwell, Arlington, TN (US); Thomas E. Drochner, Memphis, TN (US)

(73) Assignee: WARSAW ORTHOPEDIC, INC., Warsaw, IN (US)

(21) Appl. No.: 13/280,062

(22) Filed: Oct. 24, 2011

(51) Int. Cl. A61F 2/44

(52) U.S. Cl. 623/17.16

(57) ABSTRACT

An interbody implant system includes at least one endplate member defining a longitudinal axis, a trial member and a permanent implant. The endplate member is configured for engagement with a vertebral endplate and permanent implantation. The trial member is configured for disposal adjacent the at least one endplate member within an intervertebral space. The permanent implant member has a configuration and dimension corresponding to the trial member, and is disposed adjacent the at least one endplate member within the intervertebral space.

### TECHNICAL FIELD

The present disclosure generally relates to medical devices, systems and methods for the treatment of musculoskeletal disorders, and more particularly to an interbody implant system and method that provides stabilization and height restoration for treating a vertebral column while protecting the structural integrity of vertebral endplates.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. These treatments may employ interbody implants. This disclosure describes an improvement over these prior art technologies.

### SUMMARY OF THE INVENTION

Accordingly, an interbody implant system and method is provided that provides stabilization and height restoration for treating a vertebral column while protecting the structural integrity of vertebral endplates. It is contemplated that the interbody implant system and method may be employed for an arthrodesis treatment using minimally invasive and percutaneous techniques.

In one embodiment an interbody implant system comprising at least one endplate member defining a longitudinal axis, the endplate member configured for engagement with a vertebral endplate and permanent implantation is provided. The implant system having a permanent implant member having a selected configuration and being disposed adjacent the at least one endplate member within the intervertebral space. The at least one endplate member comprises a first member defining a first longitudinal axis and being engageable with a first vertebral endplate, and a second member defining a second longitudinal axis and engageable with a second opposing vertebral endplate.

In one embodiment, an interbody implant system is provided which includes at least one endplate member defining a longitudinal axis, a trial member and a permanent implant. The endplate member is configured for engagement with a vertebral endplate and permanent implantation. The trial member is configured for disposal adjacent the at least one endplate member within an intervertebral space. The permanent implant member has a configuration and dimension corresponding to the trial member, and is disposed adjacent the at least one endplate member within the intervertebral space.

In another embodiment, the interbody implant system includes a first member, a second member, a trial member and a permanent implant member. The first member is configured for alignment with a first vertebral endplate surface and permanent implantation therewith. The first member defines a longitudinal axis, a first surface engageable with the first vertebral endplate surface and a second surface oriented in an opposing direction. The second surface defines a first keyway. The second member is configured for alignment with a second vertebral endplate surface and permanent implantation therewith. The second member defines a longitudinal axis, a first surface engageable with the second vertebral endplate surface and a second surface oriented in an opposing direction. The second surface of the second member defines a second keyway. The trial member is configured for removable disposal with the first and second members between the first and second vertebral endplate surfaces. The trial member includes a first rail disposable in the first keyway and second rail disposable in the second keyway. The permanent implant member has a configuration and dimension corresponding to the trial member and is disposed with the first and second members between the first and second vertebral endplate surfaces.

In yet another embodiment, a method for treating a spine is provided. The method includes the steps of: (1) providing at least one endplate member; (2) delivering the at least one endplate member into an intervertebral space and into alignment with a vertebral endplate for permanent implantation; (3) providing at least one trial member; (4) delivering the at least one trial member adjacent the at least one endplate member within the intervertebral space; (5) selecting one of the at least one trial member to determine a configuration and dimension of the intervertebral space; (6) providing a permanent implant member based on the selected trial member; and (7) delivering the permanent implant member adjacent the at least one endplate member within the intervertebral space for permanent implantation.

### BRIEF DESCRIPTION OF THE DRAWINGS

The present disclosure will become more readily apparent from the specific description accompanied by the following drawings, in which:

FIG. 1 is a perspective view of one particular embodiment of an endplate member of a system in accordance with the principles of the present disclosure;

FIG. 2 is a perspective view of the endplate member shown in FIG. 1;

FIG. 3 is a perspective view of the endplate member shown in FIG. 1 and one embodiment of a trial member engaged with an instrument of a system in accordance with the principles of the present disclosure;

FIG. 4 is a perspective view of the trial member shown in FIG. 3;

FIG. 5 is a perspective view of a permanent endplate member of a system in accordance with the principles of the present disclosure;

FIG. 6 is a perspective view of the endplate member shown in FIG. 1 and the permanent endplate member shown in FIG. 5;

FIG. 7 is a perspective view of the endplate member shown in FIG. 1 engaged with one embodiment of an instrument of a system in accordance with the principles of the present disclosure;

FIG. 8 is a perspective view of the endplate member shown in FIG. 1 engaged with the instrument and the trial member shown in FIG. 3 engaged with the instrument shown in FIG. 5;

FIG. 9 is a perspective view of the endplate member shown in FIG. 1 engaged with the instrument shown in FIG. 3 and the permanent implant member shown in FIG. 6;

FIG. 10 is a perspective view of the endplate member shown in FIG. 1 and the permanent implant member shown in FIG. 5;

FIG. 11 is a perspective view of the endplate member shown in FIG. 1 and the permanent implant member shown in FIG. 5 with a backout prevention member in accordance with the principles of the present disclosure;

FIG. 12 is a perspective view of the endplate member shown in FIG. 1 and one embodiment of a trial member of a system in accordance with the principles of the present disclosure;

FIG. 13 is a perspective view of a first member of the endplate member shown in FIG. 1 and a retractor of a system in accordance with the principles of the present disclosure;

FIG. 14 is a perspective view of one embodiment of an endplate member and a trial member of a system in accordance with the principles of the present disclosure;

FIG. 15 is a perspective view of the endplate member shown in FIG. 14 and one embodiment of a permanent implant member of a system in accordance with the principles of the present disclosure;

FIG. 16 is a perspective view of the endplate member shown in FIG. 14 and one embodiment of a permanent implant member of a system in accordance with the principles of the present disclosure;

FIG. 17 is a perspective view of one embodiment of an endplate member of a system in accordance with the principles of the present disclosure and the permanent implant member shown in FIG. 15;

FIG. 18 is a side, cross-sectional view of the endplate member shown in FIG. 17 and the permanent implant member shown in FIG. 15; and

FIG. 19 is a perspective view of one embodiment of an endplate member and one embodiment of a permanent implant member of a system in accordance with the principles of the present disclosure.

Like reference numerals indicate similar parts throughout the figures.

### DETAILED DESCRIPTION OF THE INVENTION

The exemplary embodiments of the interbody implant system and related methods of use disclosed are discussed in terms of medical devices for the treatment of musculoskeletal disorders and more particularly, in terms of an interbody implant that provides stabilization and height restoration for treating a vertebral column while protecting the structural integrity of vertebral endplates. It is envisioned that the interbody implant system may be employed for fusion and fixation treatments to provide decompression, restoration of lordosis and resistance of subsidence into tissue, for example, vertebral endplates. It is further envisioned that the interbody implant system and methods of use disclosed can be employed to obtain fusion of vertebrae through a minimally invasive or percutaneous technique. In one embodiment, the disclosed interbody implant system and methods of use can provide improved spinal treatment with two anatomically shaped plates that may be inserted into the disc space between two adjacent vertebrae to protect the vertebral endplates during trialing, in which the proper size and/or shape of an implant to be permanently inserted into the disc space is determined. Once the trialing is complete, a permanent implant member having and the proper size and/or shape is inserted between the anatomical plates to form a final assembled implant. Assembly may occur in situ. It is contemplated that the anatomical plates may be inserted independently, such that each plate is inserted using its own insertion instrument. It is also contemplated that the anatomical plates may be connected to an insertion instrument that couples both plates together. It is further contemplated that the insertion instrument may incorporate a hook to place over a retractor blade(s) to maintain the anatomical plates in place. The hook may include a sliding and locking feature so that the length of the retractor blade(s) may be adjusted. That is the length of the retractor blades determine the placement of the hooks and where the hooks are coupled or locked in place. This feature adjusts the length of the blades.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed interbody implant system may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The present disclosure may also be alternatively employed with procedures for treating the lumbar, cervical, thoracic and pelvic regions of a spinal column. The interbody implant system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

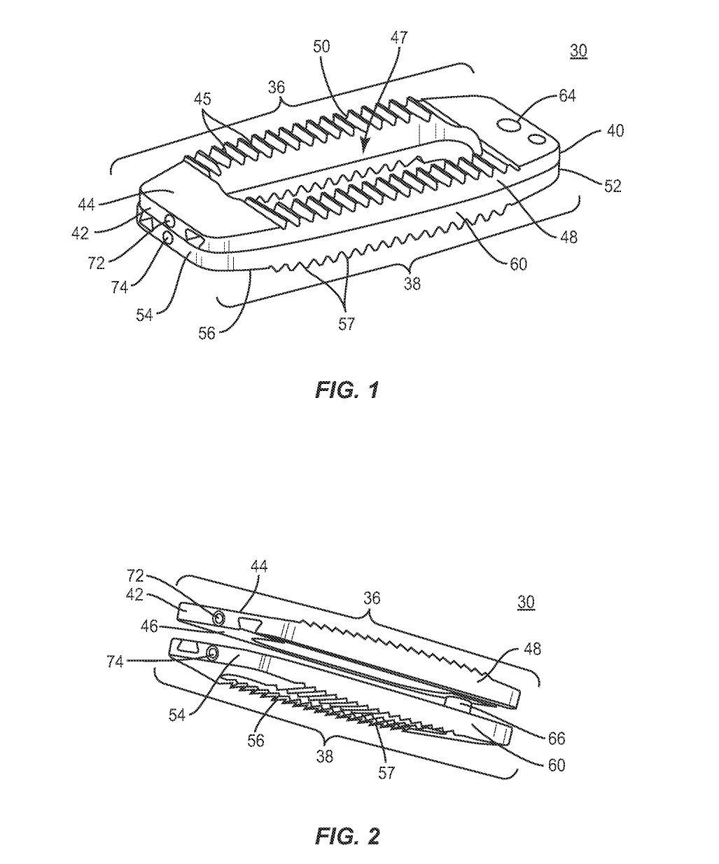

The following discussion includes a description of an interbody implant system and related methods of employing the interbody implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-6, there is illustrated components of an interbody implant system in accordance with the principles of the present disclosure.

The components of the interbody implant system can be fabricated from biologically acceptable materials suitable for medical applications, including metals, synthetic polymers, ceramics and bone material and/or their composites, depending on the particular application and/or preference of a medical practitioner. For example, the components of the interbody implant system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the interbody implant system may have material composites, including the above materials, to achieve various desired characteristics such as strength, rigidity, elasticity, compliance, biomechanical performance, durability and radiolucency or imaging preference. The components of the interbody implant system, individually or collectively, may also be fabricated from a heterogeneous material such as a combination of two or more of the above-described materials. For example, in one embodiment, the at least one endplate member 30 may be fabricated from titanium or titanium alloy, while the permanent implant member 34 may be fabricated from PEEK and/or PEEK composites.

The interbody implant system includes at least one endplate member 30, a trial member 32 and a permanent implant member 34. Endplate member 30 is anatomically configured for engagement and alignment with a vertebral endplate and permanent implantation thereto. In one embodiment, endplate member 30 includes a first member 36 defining a first longitudinal axis engageable with a first vertebral endplate and a second member 38 defining a second longitudinal axis engageable with a second opposing vertebral endplate. It is envisioned that the first and second longitudinal axes may be the same such that first and second members 36, 38 are aligned with one another. It is also envisioned that the first and second longitudinal axes may be different such that first and second members 36, 38 are disposed at an angle relative to one another.

First member 36 includes a proximal end 40, a distal end 42 opposite proximal end 40, upper and lower surfaces 44, 46, and side surfaces 48, 50. Upper surface 44 is configured to interface with a load bearing endplate of a vertebra and is convexly curved between proximal end 40 and distal end 42 such that the height of first member 36 at a point between proximal end 40 and distal end 42 is greater than the height of first member 36 at proximal end 40 and/or distal end 42. However, it is envisioned that upper surface 44 may also be planar along the length thereof such that upper surface 44 has a uniform height between proximal end 40 and distal end 42. Upper surface 44 may include bone engaging features 45 configured to reduce slipping or movement relative to the endplate of the vertebra upper surface 44 is configured to interface. Lower surface 46 is planar along the length thereof and is configured to interface with a corresponding surface of trial member 32 or permanent implant member 34. First member 36 has a width defined by the distance between side surfaces 48, 50 that is approximately the width of the endplate of the vertebra first member 36 is configured to interface. In some embodiments, the width defined by the distance between the side surfaces 48, 50, is less than the approximate width of the endplate of the vertebra first member 36 is configured o interface. First member 36 has a length defined by the distance between proximal end 40 and distal end 42 which is approximately the length of the endplate of the vertebra first member 36 is configured to interface. First member 36 may include an Opening 47 extending through upper and lower surfaces 44, 46 between side surfaces 48, 50.

Second member 38 has a configuration similar to first member 36 and includes a proximal end 52, a distal end 54 opposite proximal end 52, upper and lower surfaces 56, 58, and side surfaces 60, 62. Upper surface 56 is configured to interface with a load bearing endplate of a vertebra adjacent the endplate of the vertebra upper surface 44 of first member 36 is configured to interface and is convexly curved between proximal end 52 and distal end 52 such that the height of second member 38 at a point between proximal end 52 and distal end 54 is greater than the height of second member 38 at proximal end 52 and/or distal end 54. However, it is envisioned that upper surface 56 may also be planar along the length thereof such that upper surface 56 has a uniform height between proximal end 52 and distal end 54. Upper surface 56 may include bone engaging features 57 configured to reduce slipping or movement relative to the endplate of the vertebra upper surface 56 is configured to interface. Lower surface 58 is planar along the length thereof and is configured to interface with a corresponding surface of trial member 32 or permanent implant member 34. Second member 38 has a width defined by the distance between side surfaces 60, 62 that is approximately the width of the endplate of the vertebra second member 38 is configured to interface. Second member 38 has a length defined by the distance between proximal end 52 and distal end 54, which is approximately the length of the endplate of the vertebra second member 38, is configured to interface. Second member 38 may include an opening (not shown) extending through upper and lower surfaces 56, 58 between side surfaces 60, 62. The opening in second member 38 is configured to align with opening 47 in first member 36 such that the opening in second member 38 is in communication with opening 47.

In one embodiment, first member 36 includes a first locking part, such as, for example, a cavity 64 disposed in proximal end 40 between side surfaces 48, 50. Cavity 64 is disposed equidistant between side surfaces 48, 50. However, it is envisioned that cavity 64 may be disposed closer to side surface 48 than side surface 50, or vice versa. Cavity 64 extends through upper and lower surfaces 44, 46 such that cavity 64 defines an opening in upper and lower surfaces 44, 46 defining a passageway therethrough. However, it is envisioned that cavity 64 may extend through lower surface 46 without extending through upper surface 44. Cavity 64 is configured to receive a corresponding locking part of second member 38 to couple first and second members 36, 38 to one another.

Second member 38 includes a second coupling or locking part, such as, for example, a protrusion 66 disposed in proximal end 52 between side surfaces 60, 62 configured to be received within cavity 64. It is understood that although the term locked is used it includes embodiments where the parts are simply coupled together. Protrusion 66 is positioned equidistant between side surfaces 60, 62. However, it is envisioned that protrusion 66 may be positioned such that protrusion 66 is closer to side surface 60 than side surface 62, or vice versa. Protrusion 66 extends perpendicularly from lower surface 58 of second member 38 a distance such that trial member 32 or permanent implant member 34 may be placed between first and second members 36, 38 when protrusion 66 is received within cavity 64 to couple first and second members 36, 38 to one another. In one embodiment, cavity 64 and protrusion 66 have corresponding cylindrical cross-sections, such that protrusion 66 may be received within cavity 64. However, it is envisioned that cavity 64 and/or protrusion 66 may have other corresponding cross-sectional configurations, such as, for example, oval, oblong, triangular, rectangular, square, polygonal, irregular, uniform, non-uniform, variable and/or tapered. In one embodiment, protrusion 66 is moveably disposed in cavity 64 such that first and second members 36, 38 may pivot relative to one another to separate distal ends 42, 54 of first and second members 36, 38 to allow trial member 32 or permanent implant member 34 to be inserted between first and second members 36, 38. For example, protrusion 66 may include a ball at an end thereof configured to be received within a corresponding cavity 64 such that protrusion 66 can rotate within cavity 64. It is envisioned that first member may pivot axially about protrusion 66 relative to second member 38 such that first member 36 may be off-set from second member 38 at an angle. In one embodiment, protrusion 66 is fixed within cavity 64 in a configuration to align first and second members 36, 38. It is envisioned that protrusion 66 and cavity 64 could be positioned on both the first and second members so that both members have a protrusion and a cavity that couples them together.

First and second members 36, 38 are configured to be inserted in between the endplates of adjacent vertebrae to protect the endplates from undesirable engagement of a trial member with a vertebral endplate during trialing, in which various trial members, such as trial member 32, may be inserted between first and second members 36, 38 to determine the proper size and shape of an intervertebral space and hence an intervertebral implant. After the size and shape of the intervertebral space is determined, the trial member may be removed and a permanent implant member having the proper size and shape, such as permanent implant member 34, may be inserted in place of the trial member.

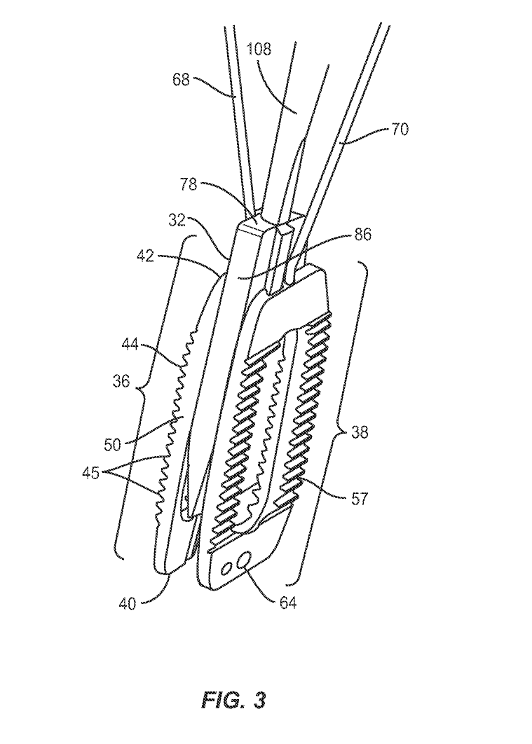

One or more instruments, such as instruments 68, 70, may be used to facilitate insertion of first member 36 and/or second member 38 into an intervertebral space. As discussed above, it is envisioned that first and second members 36, 38 may be inserted into an intervertebral space individually, or coupled to one another. As shown in FIG. 3, instruments 68, 70 are each defined by an elongate section, instrument 68 being engaged with first member 36 and instrument 70 being engaged with second member 38. In particular, first member 36 includes a recess 72 disposed in distal end 42 of first member 36 equidistant between side surfaces 48, 50. In one embodiment according to the principles of the present disclosure, recess 72 and 74 are positioned off-center between side surfaces 48 and 50. Recess 72 is configured to receive an end of instrument 68 or instrument 70 such that instrument 68 or instrument 70 may be used to manipulate first member 36 for insertion into an intervertebral space. Likewise, second member 38 includes a recess 74 disposed in distal end 54 of second member 38 equidistant between side surfaces 60, 62. Recess 74 is configured to receive an end of instrument 68 or instrument 70 such that instrument 68 or instrument 70 may be used to manipulate second member 38. Instruments 68, 70 may have an elongated section, which is straight along the length thereof, or may have at least one bend along the length thereof In one embodiment, instruments 68, 70 may include a U-joint along the length of the elongated section such that one end of the elongated section may pivot in any direction relative to the opposite end of the elongated section. In one embodiment, instruments 68, 70 may each include a U-joint at one end of the elongated section, at least a portion of the U-joint being configured to be received within recesses 72, 74.

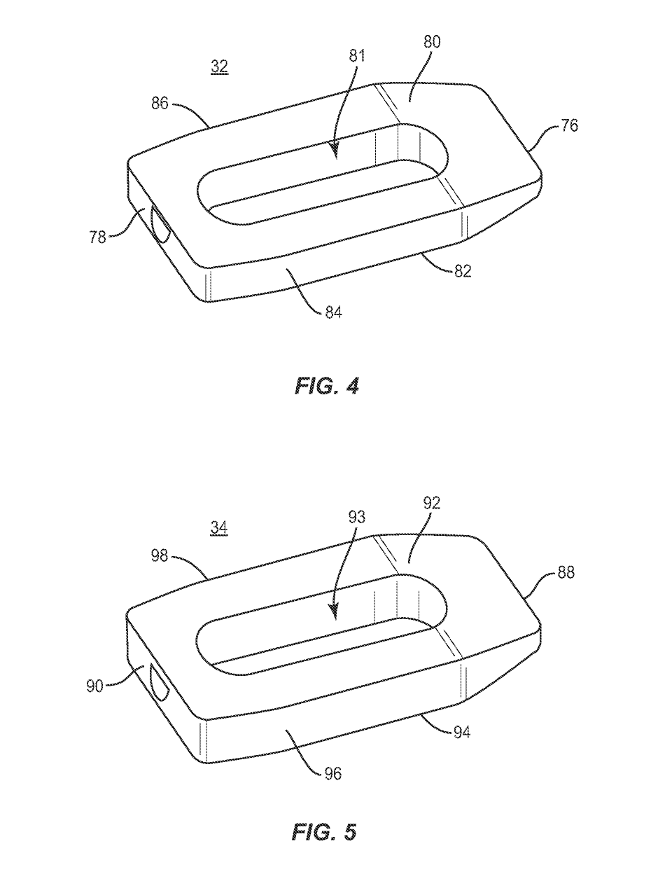

Trial member 32 is configured for disposal adjacent endplate member 30 within an intervertebral space to determine the height of the intervertebral space and/or lordotic angle. As best shown in FIG. 4, trial member 32 includes a proximal end 76, a distal end 78 opposite proximal end 76, upper and lower surfaces 80, 82, and side surfaces 84, 86. Upper surface 80 is configured to interface with lower surface 46 of first member 36 and is planar between proximal end 76 and distal end 78; lower surface 82 is configured to interface with lower surface 58 of second member 38 and is planar between proximal end 76 and distal end 78. That is, the height of trial member 32 is uniform between proximal end 76 and/or distal end 78. However, it is envisioned that the height of trial member 32 corresponds to the geometry of an intervertebral space and therefore may also be non-uniform. Proximal end 76 and/or distal end 78 may be tapered such that the height of trial member 32 is less at proximal end 76 and/or distal end 78 than the height at a point between proximal and distal ends 76, 78. The amount of taper is dependent upon, for example, the curve between two adjacent vertebrae, such as, for example, the angles of lordosis. This tapered confirmation is designed to ease insertion into the disc space between the endplate members 36 and 38. The height of trial member 32 is approximately the distance between the endplates of the adjacent vertebrae, which define an intervertebral space less the height of first and second members 36, 38. That is, first and second members 36, 38 and trial member 32 combine to occupy the intervertebral space between two adjacent vertebrae. In one embodiment, in which only first member 36 or second member 38 is inserted into an intervertebral space, the height of trial member 32 is approximately the distance between the endplates of the adjacent vertebrae which define an intervertebral space less the height of first member 36 or second member 38. Trial member 32 has a width defined by the distance between side surfaces 84, 86 that is approximately the width of first member 36 and/or second member 38. Trial member 32 has a length defined by the distance between proximal end 76 and distal end 78, which is approximately the length of first member 36 and/or second member 38. Trial member 32 may include an opening 81 extending through upper and lower surfaces 80, 82 between side surfaces 84, 86. Opening 87 may align with opening 47 in first member 36 and the opening in second member 38 such that all three openings are in communication. That is, the three openings define a passageway through first member 36, trial member 32 and second member 38.

It is envisioned that the system of the present disclosure may include a plurality of trial members, each having differing heights and angles of lordosis, to determine the geometry of a permanent implant member that should be inserted between first and second members 36, 38. In one embodiment, distal end 78 is configured to engage an instrument, such as instrument 108, to manipulate trial member 32 such that trial member 32 can be inserted and/or removed from a location adjacent first and second members 36, 38 within an intervertebral space. In one embodiment, instrument 108 could be configured so that the trial member 32 is permanently attached to the instrument 108.

Trial member 32 is configured to be inserted between first and second members 36, 38 of endplate member 30. When first and second members 36, 38 are coupled to one another by inserting protrusion 66 of second member 38 into cavity 64 of first member 36, trial member 32 may be inserted between distal ends 42, 54 of first and second members 36, 38 leading with proximal end 76 and is advanced proximally until proximal end 76 engages protrusion 66 of second member 38, which prevents trial member 32 from advancing further.

Permanent implant member 34 has a configuration and dimension corresponding to trial member 32, and is disposed adjacent first and second members 36, 38 of endplate member 30 within an intervertebral space. As best shown in FIG. 5, permanent implant member 34 includes a proximal end 88, a distal end 90 opposite proximal end 88, upper and lower surfaces 92, 94, and side surfaces 96, 98. Upper surface 92 is configured to interface with lower surface 46 of first member 36 and is planar between proximal end 88 and distal end 90; lower surface 94 is configured to interface with lower surface 58 of second member 38 and is planar between proximal end 88 and distal end 90. That is, the height of permanent implant member 34 is uniform between proximal end 88 and/or distal end 90. However, it is envisioned that the height of permanent implant member 34 corresponds to the geometry of an intervertebral space and therefore may also be non-uniform. Proximal end 88 and/or distal end 90 may be tapered such that the height of permanent implant member 34 is less at proximal end 88 and/or distal end 90 than the height at a point between proximal and distal ends 88, 90. The amount of taper is dependent upon, for example, the curve between two adjacent vertebrae, such as, for example, the angles of lordosis or concavity of vertebral endplates.

The height of permanent implant member 34 is approximately the distance between the endplates of the adjacent vertebrae, which define an intervertebral space less the height of first and second members 36, 38. That is, first and second members 36, 38 and permanent implant member 34 combine to occupy the intervertebral space between two adjacent vertebrae. In one embodiment, in which only first member 36 or second member 38 is inserted into an intervertebral space, the height of permanent implant member 34 is approximately the distance between endplates of the adjacent vertebrae which define an intervertebral space less the height of first member 36 or second member 38. Permanent implant member 34 has a width defined by the distance between side surfaces 96, 98 that is approximately the width of first member 36 and/or second member 38. Permanent implant member 34 has a length defined by the distance between proximal end 88 and distal end 90, which is approximately the length of first member 36 and/or second member 38. Permanent implant member may include an opening 93 extending through upper and lower surfaces 92, 94 between side surfaces 96, 98 configured for disposal of a material, such as, for example, a bone growth material. Opening 93 may align with opening 47 in first member 36 and the opening in second member 38 such that all three openings are in communication. That is, the three openings define a passageway through first member 36, permanent implant member 34, and second member 38 such that a material disposed in opening 93 may reach the endplates through opening 47 and/or the opening in second member 38.

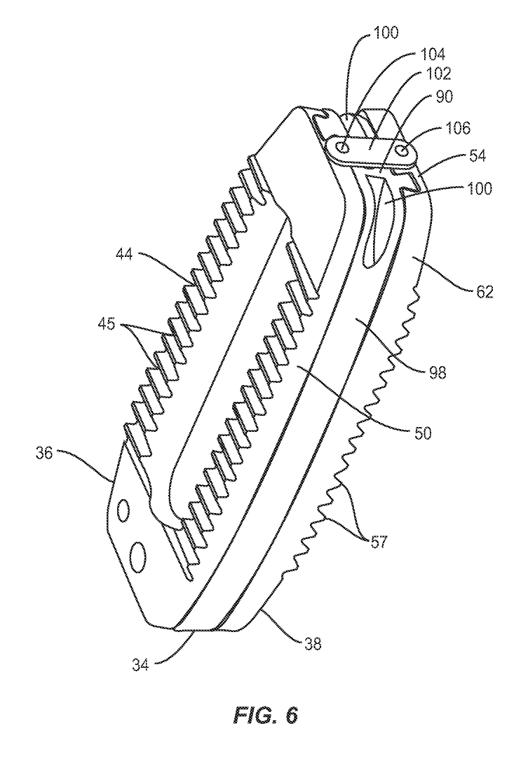

In one embodiment, distal end 90 of permanent implant member 34 is configured to engage an instrument to manipulate permanent implant member 34 such that permanent implant member 34 can be inserted and/or removed from a space between first and second members 36, 38, when first and second members 36, 38 are inserted in an intervertebral space. It is envisioned that permanent implant member 34 may include at least one recess 100 disposed in distal end 90 thereof configured to receive at least a portion of an instrument, as shown in FIG. 6. Recess 100 extends from distal end 90 toward proximal end 88. Recess 100 may extend through side surface 96 and/or side surface 98. It is also envisioned that recess 100 may be disposed in distal end 90 of permanent implant member 34 without extending through side surface 96 and/or side surface 98.

Permanent implant member 34 is configured to be inserted between first and second members 36, 38 of endplate member 30 when first and second members 36, 38 are coupled to one another by inserting protrusion 66 of second member 38 into cavity 64 of first member 36, permanent implant member 34 may be inserted between distal ends 42, 54 of first and second members 36, 38 leading with proximal end 88 and is advanced proximally until proximal end 88 engages protrusion 66 of second member 38, which prevents permanent implant member 34 from advancing further. Permanent implant member 34 may then be positioned by moving permanent implant member 34 relative to first member 36 and/or second member 38 such that permanent implant member 34 is aligned with first member 36 and/or second member 38.

In one embodiment of the system of the present disclosure, a backout prevention member 102 is provided to maintain permanent implant member 34 in position between first and second members 36, 38, while maintaining the position of first member 36 relative to second member 38. Backout prevention member 102 is a tab which extends transversely across distal ends 42, 54, 90 of first member 36, second member 38 and permanent implant member 34 and includes a protrusion 104 configured to be received within recess 72 of first member 36 and a protrusion 106 configured to be received within recess 74 of second member 38. In one embodiment, protrusions 104 are separate screws or pins configured to lock the backout preventer member 102 in place. That is, at least a portion of protrusions 104, 106 are inserted into recesses 72, 74 to lock backout prevention member 102 in place which maintains first and second members 36, 38 in position relative to one another, and prevents permanent implant member 34 from backing out from its location between first and second members 36, 38. It is envisioned that backout prevention member 102 may be used to maintain a trial member, such as trial member 32, in position between first and second members 36, 38, while maintaining the position of first member 36 relative to second member 38.

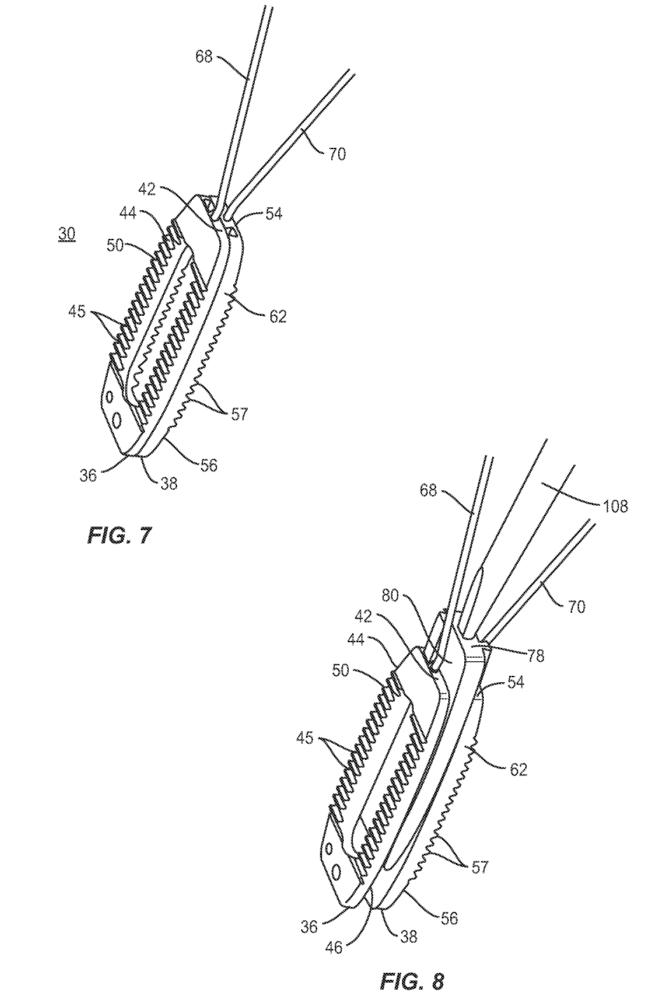

As discussed above, endplate member 30 is configured to be inserted between the endplates of adjacent vertebrae. Prior to insertion, first and second members 36, 38 of endplate member 30 may be coupled to one another, as shown in FIG. 7. That is, first and second members 36, 38 may be inserted into an intervertebral space individually or coupled together as a single unit. First and second members 36, 38 may be coupled to one another by inserting protrusion 66 of second member 38 into cavity 64 of first member 36, such that first member 36 may pivot relative to second member 38 to align first and second members 36, 38. Endplate member 30, having first and second members 36, 38 aligned with one another, may then be delivered within an intervertebral space, between the endplates of adjacent vertebrae in order to protect the endplates. It is also envisioned that a surgeon may align first and second members 36, 38 with one another after endplate member 30 is delivered within the intervertebral space. Instruments 68, 70, may be used to facilitate insertion of first and second members 36, 38 into the intervertebral space and/or alignment of first and second members 36, 38 with the endplates for permanent implantation. It is envisioned that instruments 68, 70 may connect to first and second members 36, 38 at an angular orientation relative to a longitudinal axis of endplate member 30.

Once endplate member 30 is positioned within the intervertebral space, trial member 32 is then delivered adjacent endplate 30 member within the intervertebral space such that upper surface 80 of trial member 32 interfaces with lower surface 46 of first member 36 and lower surface 82 of trial member 32 interfaces with lower surface 58 of second member 38, as shown in FIG. 8. Proximal end 76 of trial member 32 may be tapered to facilitate insertion of trial member 32 between first and second members 36, 38. As discussed above, distal end 78 of trial member 32 is configured to engage instrument 108 to insert and/or remove trial member 32 adjacent endplate 30 member within the intervertebral space. The surgeon will then determine if endplate member 30 and trial member 32 provide a height, shape and angles of lordosis that correspond to the geometry of the intervertebral space and/or the endplates of the vertebrae. The surgeon may insert trial members that differ as to size and shape until the configuration and dimension of the intervertebral space is determined. At which time, trial member 32 may be removed from its position between first and second members 36, 38.

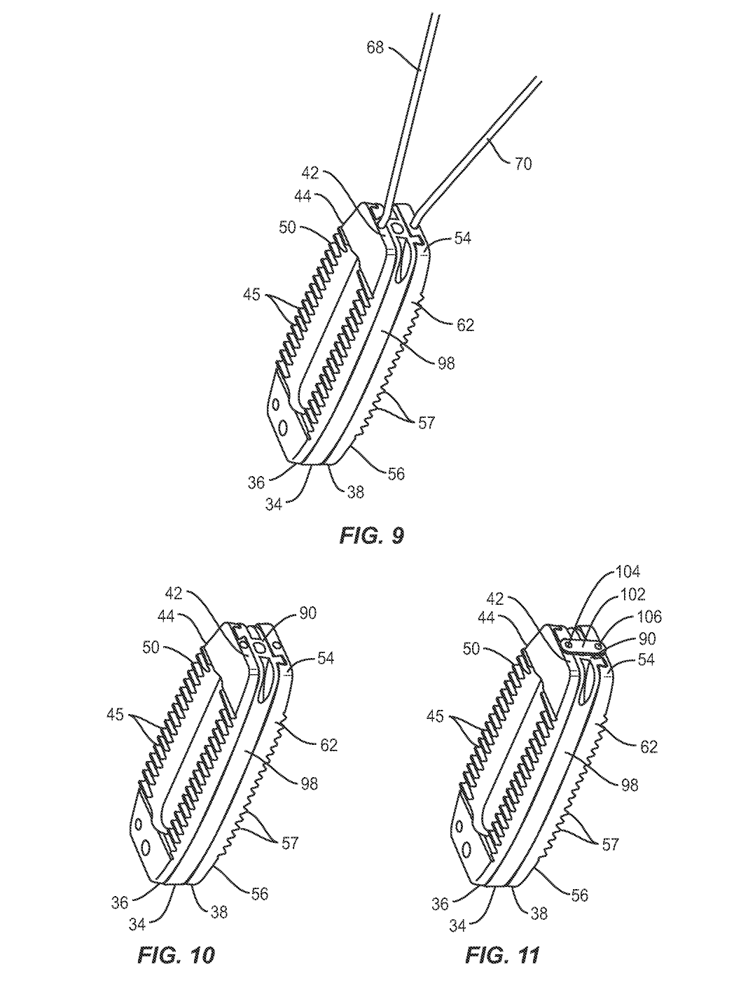

Permanent implant member 34 having a size and shape which corresponds to that of the selected trial member 32 may then be delivered adjacent endplate member 30 within the intervertebral space for permanent implantation. In particular, permanent implant member 34 is inserted between first and second members 36, 38 after trial member 32 is removed therefrom, as shown in FIG. 9. Following insertion of permanent implant member 34, instruments 68, 70 used to manipulate first and second members 36, 38 and/or any instrument(s) used to manipulate permanent implant member 34 may be removed, as shown in FIG. 10. Backout prevention member 102 may then be attached by inserting protrusions 104, 106 into recesses 72, 74 to maintain permanent implant member 34 in position between first and second members 36, 38, while maintaining the position of first member 36 relative to second member 38, as shown in FIG. 11.

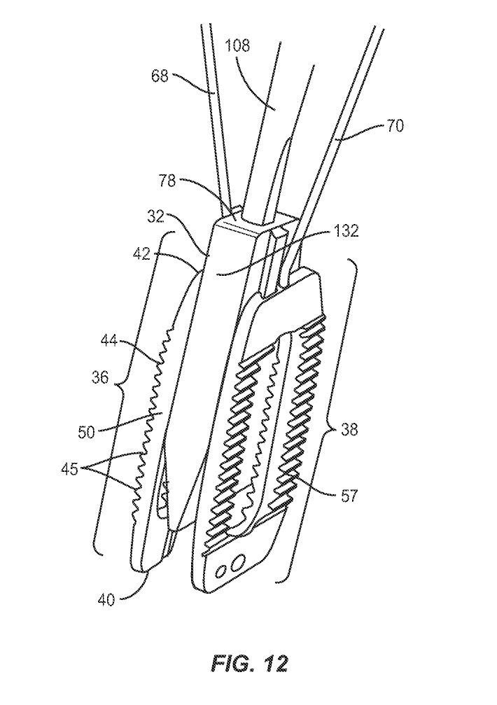

As discussed above, the system of the present disclosure may include a plurality of trial members, similar to trial member 32, but having differing heights and angles of lordosis, to determine the height of a permanent implant member that should be inserted between first and second members 36, 38. Shown in FIG. 12 is a trial member 132 having a configuration similar to that of trial member 32, but has a height that is greater than that of trial member 32. Furthermore, the amount of taper in the proximal end of trial member 132 is greater than that in proximal end 76 of trial member 32.

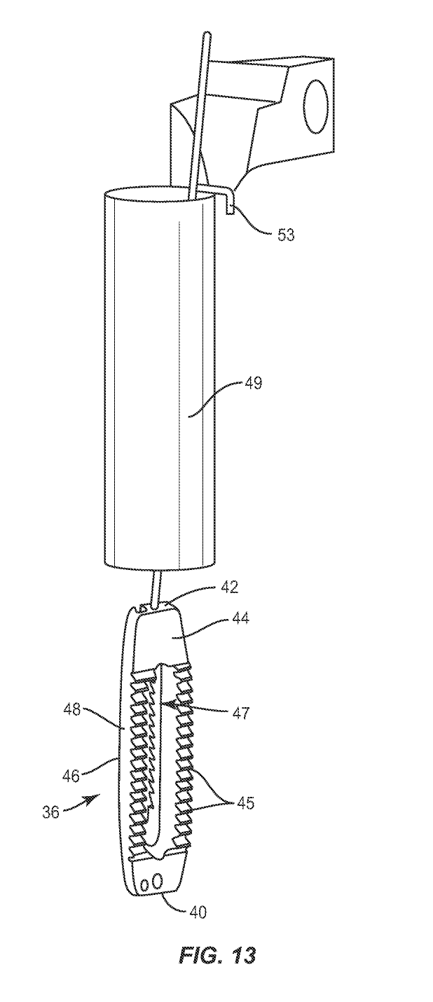

In one embodiment, as shown in FIG. 13, the first or second member 36, 38 is configured so that the instrument 68 or 70 has a hook portion at the distal end that extends outside the patient. Hook 53 is used to connect the instrument 68, 70 with the first or second members 36, 38 or the retractor 49 so as to temporarily free the hands of the surgeon.

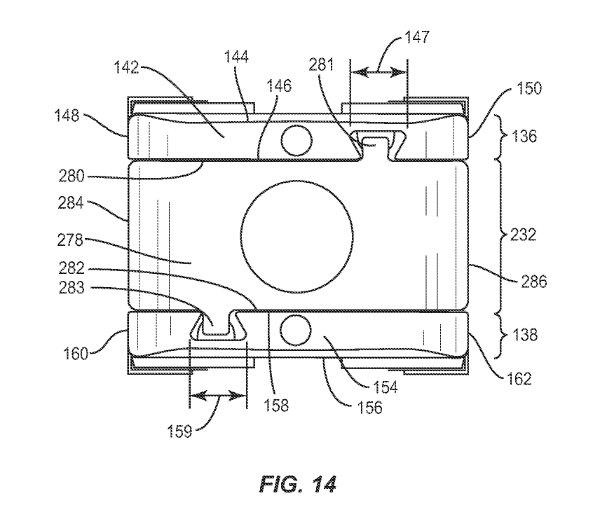

In one embodiment, shown in FIG. 14, the interbody implant system includes a trial member 232 having a configuration similar to that of trial members 32, 132, and first and second members 136, 138 each having a configuration similar to that of first and second member 36, 38. Trial member 232 includes a distal end 278, a proximal end (not shown) opposite distal end 276, upper and lower surfaces 280, 282, and side surfaces 284, 286. Upper surface 280 has a first rail 281 extending therefrom; lower surface 282 has a second rail 283 extending therefrom. First rail 281 is configured to be disposed in a keyway extending through first member 136 and second rail 283 is configured to be disposed in a keyway extending through second member 138. It is envisioned that first and second rails 281 and 283 extend away from the upper and lower surfaces in a perpendicular orientation or at an acute or obtuse angle. The keyways configured to accommodate of the first and second rails 281, 283.

In one embodiment, first and second rails 281, 283 each extend from distal end 278 of trial member 232 to the proximal 1 end thereof However it is envisioned that first rail 281 may be extend along a portion of upper surface 280 between distal end 278 and the proximal end of trial member 232. Likewise, second rail 283 may extend along a portion of lower surface 282 between distal end 278 and the proximal end of trial member 232. In one embodiment, first and second rails 281, 283 each have a planar top portion between arcuate side surfaces. First and second rails 281, 283 each taper from the top portion of each rail to the surface of trial member 232 from which each rail extends such that the top portions of first and second rails 281, 283 each have a width which is greater than the width of first and second rails 281, 283 adjacent the surface of trial member 232 from which they extend. That is, first and second rails 281, 283 each have a T-shaped cross-section. However, it is envisioned that first and second rails 281, 283 may have cross-sections corresponding the size and shape of the keyways in first and second members 136, 138, including, for example, L-shaped, oval, oblong, triangular, rectangular, square, polygonal, irregular, uniform, non-uniform, variable and/or tapered. In one embodiment, first and second rails 281, 283 are offset. That is, first rail 281 is disposed closer to side surface 286 than side surface 284 and second rail 283 is disposed closer to side surface 284 than side surface 286. However, it is envisioned that first rail 281 may be disposed closer to side surface 284 than side surface 286 and second rail 283 may be disposed closer to side surface 286 than side surface 284. Alternatively, first and second rails 281, 283 may be equidistant from side surfaces 284, 286.

First member 136 includes a first keyway 147 configured to receive first rail 281 and second member 138 includes a second keyway 159 configured to receive second rail 283. First and second keyways 147, 159 have cross-sections corresponding to the size and shape of first and second rails 281, 283, including, for example, oval, oblong, triangular, rectangular, square, polygonal, irregular, uniform, non-uniform, variable and/or tapered. Since the trial is used to determine the size of the implant that needs to go between the endplate members the keyways and rails do not necessarily need to perfectly match each other. In one embodiment, the endplate members can be lifted off the trial without the rails interfering. The rails are provided fir side-to-side positioning so as to facilitate easy permanent implant insertion. In one embodiment, first and second keyways 147, 149 have a diameter at the top of each keyway which is greater than the diameter at bottom of each keyway, the top of each keyway having a diameter at the top of each keyway which is greater than the diameter of the top portions of first and second rails 281, 283 and a diameter at the bottom of each keyway that is less than the diameter of the top portions of first and second rails 281, 283 such that the top portions of first and second rails 281, 283 are prevented from falling through the bottom of each keyway. First keyway 147 extends along the length of first member 136 through lower surface 146 of first member 136 without extending through upper surface 144. Second keyway 159 extends along the length of second member 138 through lower surface 158 of second member 138 without extending through upper surface 156.

First and second keyways 147, 159 are offset such that the position of first keyway 147 corresponds to the position of first rail 281 and the position of second keyway 159 corresponds to the position of second rail 283 when first and second members 136, 138 are aligned with trial member 232. That is, first and second members 136, 138 and trial member 232 are configured such that first rail 281 is received within first keyway 147 and second rail 283 is received within second keyway 159 when first and second members 136, 138 are aligned with trial member 232. In particular, trial member 232 is inserted between the distal ends 142 & 154 of first and second members 136, 138 leading with the proximal end 276 such that first rail 281 is received within first keyway 147 and second rail 283 is received within second keyway 159. Trial member 232 is then advanced toward proximal ends 140, 152 with first and second members 136, 138 aligned with trial member 232.

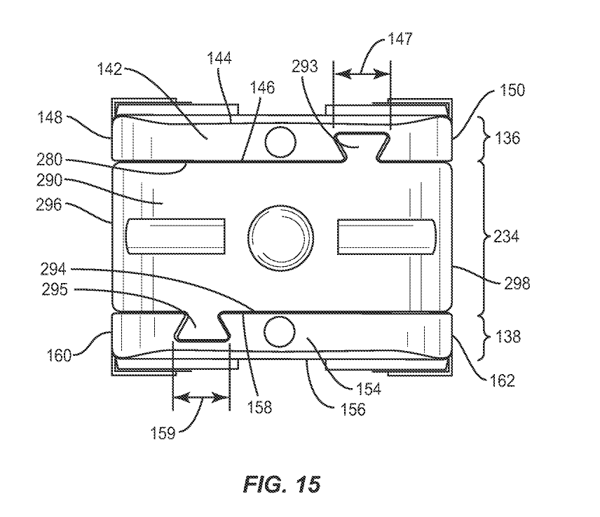

In one embodiment, shown in FIG. 15, the interbody implant system includes a permanent implant member 234 having a configuration similar to permanent implant member 34. Permanent implant member 234 includes a distal end 290, a proximal 288 end (not shown) opposite distal end 290, upper and lower surfaces 292, 294, and side surfaces 296, 298. Upper surface 292 has a first rail 293 extending perpendicularly therefrom; lower surface 294 has a second rail 295 extending perpendicularly therefrom. First rail 293 is configured to be disposed in keyway 147 and second rail 295 is configured to be disposed in keyway 159.

In one embodiment, first and second rails 293, 295 each extend from distal end 290 of permanent implant member 234 to the proximal end thereof However it is envisioned that first rail 293 may be extend along a portion of upper surface 292 between distal end 290 and the proximal end of permanent implant member 234. Likewise, second rail 295 may extend along a portion of lower surface 294 between distal end 290 and the proximal end of permanent implant member 234. In one embodiment, first and second rails 293, 295 each have a planar top portion between arcuate side surfaces. First and second rails 293, 295 each taper from the top portion of each rail to the surface of permanent implant member 234 from which each rail extends such that the top portions of first and second rails 293, 295 each have a width which is greater than the width of first and second rails 293, 295 adjacent the surface of permanent implant member 234 from which they extend. That is, first and second rails 293, 295 each have a T-shaped cross-section or dovetail cross-section. However, it is envisioned that first and second rails 293, 295 may have cross-sections corresponding the size and shape of the keyways in first and second members 136, 138, including, for example, L-shaped, oval, oblong, triangular, rectangular, square, polygonal, irregular, uniform, non-uniform, variable and/or tapered.

In one embodiment, first and second rails 293, 295 are offset. That is, first rail 293 is disposed closer to side surface 298 than side surface 296 and second rail 295 is disposed closer to side surface 296 than side surface 298. However, it is envisioned that first rail 293 may be disposed closer to side surface 296 than side surface 298 and second rail 295 may be disposed closer to side surface 298 than side surface 296. Alternatively, first and second rails 293, 295 may each be equidistant from side surfaces 296, 298. Permanent implant member 234 is inserted between the distal ends 142, 154 of first and second members 136, 138 leading with the proximal end 288 such that first rail 293 is received within first keyway 147 and second rail 295 is received within second keyway 159. Permanent implant member 234 is then advanced toward proximal ends 140, 152 with first and second members 136, 138 aligned with permanent member 234.

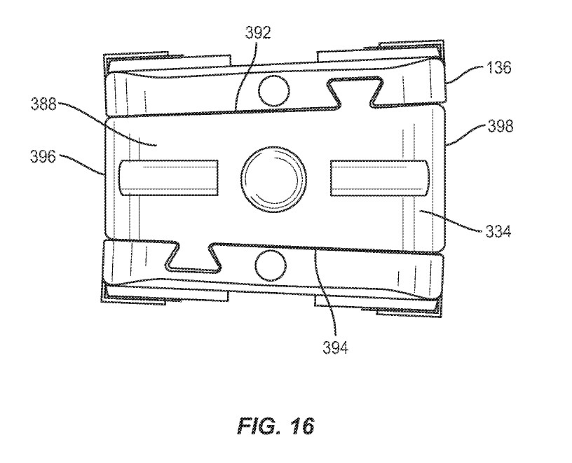

In one embodiment, shown in FIG. 16, a permanent implant member 334 is provided which has a similar configuration to permanent implant member 234. However, permanent implant member 334 has a wedge-shaped configuration to conform to the lordotic angle of the vertebrae the interbody implant system is configured to be inserted between. That is, permanent implant member 334 has a height between upper and lower surfaces 392, 394 which is greater adjacent side surface 396 than adjacent side surface 398 and decreases gradually between side surface 396 and side surface 398. It is envisioned that the height of permanent implant member 334 and/or the slope of upper surface 392 and/or lower surface 394 may be varied depending upon, for example, the height and lordotic angle of the vertebrae the interbody implant system is inserted between. It is also envisioned that the height between upper and lower surfaces 392, 394 may be greater adjacent a proximal end 388 than adjacent a distal end (not shown) or vice versa, depending upon the geometry of the intervertebral space.

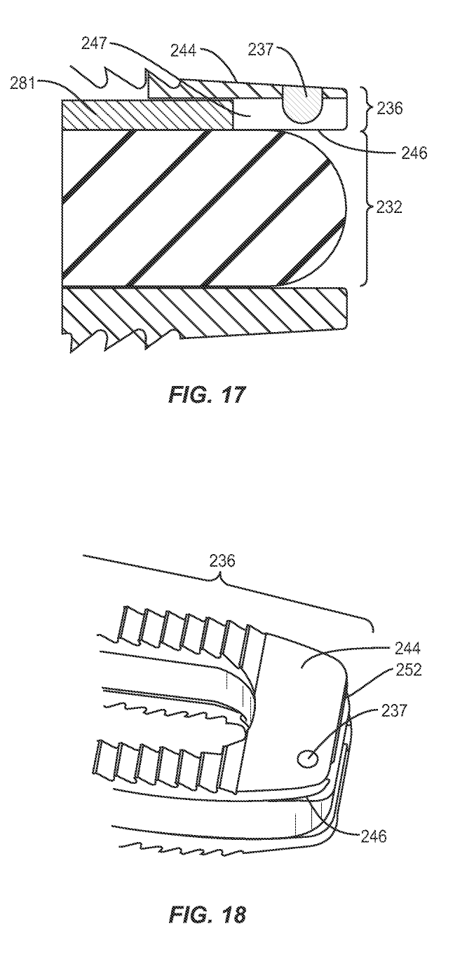

In one embodiment, shown in FIGS. 17 and 18, a first member 236 is provided having a configuration similar to that of first member 136, but includes a stop, such as, for example, a peg 237 disposed in a proximal end 240 of first member 236 configured to prevent movement of a trial member, such as trial member 232, or a permanent implant member, such as permanent implant members 234, 334 within a first keyway 247 configured to receive first rail 281 of trial member 232 and/or first rail 293 of permanent implant member 234 to align first member 236 and trial member 232 or first member 236 and permanent implant member 234. Peg 237 extends from an upper surface 244 of first member 236 transversely toward a lower surface 246 of first member 236 such that at least a portion of peg 237 is received within first keyway 247 to allow peg 237 to contact first rail 281 and/or first rail 293 and prevent the same from advancing proximally past peg 237 along first keyway 247.

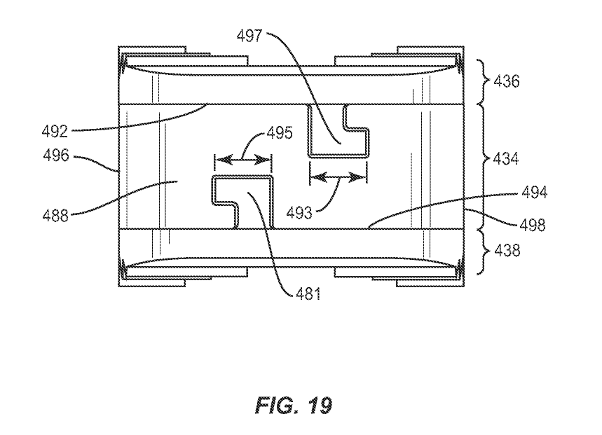

In one embodiment, shown in FIG. 19, the interbody implant system includes a permanent implant member 434 having a configuration similar to that of permanent implant members 234, 334, and first and second members 436, 438 each having a configuration similar to first and second members 136, 138. Permanent implant member 434 includes a proximal end 488, a distal end (not shown) opposite proximal end 488, upper and lower surfaces 492, 494, and side surfaces 496, 498. Upper and lower surfaces 494, 496 include first and second keyways 493, 495 configured to receive a rail 497of first member 436 and a rail 481 of second member 438. First and second keyways 493, 495 have cross-sections corresponding the size and shape of the rails which are received therein. As shown in FIG. 19, first and second keyways 493, 495 have an L-shaped cross-section. However, it is envisioned that first and second keyways 493, 495 may have cross-sections corresponding the rails which are received therein, including, for example, T-shaped, oval, oblong, triangular, rectangular, square, polygonal, irregular, uniform, non-uniform, variable and/or tapered or dove tailed. First and second keyways 493, 495 extend from proximal end 488 through the distal end of permanent implant member 434.

Second member 438 includes rail 481 extending perpendicularly therefrom configured to be disposed in second keyway 495. Rail 481has a cross-sectional configuration corresponding the size and shape of second keyway 495. It is envisioned that first member 436 may include a rail 497 extending perpendicularly therefrom configured to be disposed in first keyway 493. Accordingly, rails may extend from a trial member, as shown in FIG. 14, a permanent implant member, as shown in FIGS. 15 and 16, or from an endplate member, as shown in FIG. 19. Likewise, keyways may be disposed in an endplate member, as shown in FIGS. 14-16, or in a permanent implant member, as shown in FIG. 19.

In assembly, operation and use, the interbody implant system is employed with a surgical procedure such as a fusion treatment of a spine of a patient including vertebrae, intervertebral disc space, as discussed herein. The interbody implant system may also be employed with other surgical procedures, such as, for example, discectomy, laminotomy, laminectomy, nerve root retraction, foramenotomy, facetectomy, decompression, and spinal, nucleus or disc replacement.

For example, the interbody implant system can be employed with a surgical procedure to provide height restoration between vertebral bodies for treatment of an applicable condition or injury of an affected section of a spinal column and adjacent areas within a body, such as, for example, intervertebral disc space between the endplates of adjacent vertebrae. It is contemplated that the interbody implant system can be inserted within an intervertebral space-to-space apart articular joint surfaces, provide support and maximize stabilization of the vertebrae.

It is envisioned that the interbody implant system can be used in any existing surgical method or technique including open surgery, mini-open surgery, minimally invasive surgery and percutaneous surgical implantation, whereby vertebrae are accessed through a mini-incision, or sleeve that provides a protected passageway to the area. Once access to the surgical site is obtained, the particular surgical procedure is performed for treating the spine disorder. The interbody implant system is then employed to augment the surgical treatment. It is contemplated that one or all of the components of the interbody implant system can be delivered to the surgical site via manual manipulation and/or a free hand technique. It is further contemplated that the components of the interbody implant system may be inserted posteriorly, and then manipulated anteriorly and/or lateral and/or medial.

It is envisioned that the components of the interbody implant system can be delivered to the surgical site via alternate approaches, such as, for example, delivery through the surgical pathway along a direct lateral approach, a transforaminal lumbar interbody fusion approach and a posterior lumbar interbody fusion.

In one embodiment, the interbody implant system includes at least one agent, which may be disposed, packed or layered within, on or about the components and/or surfaces thereof. For example, at least one agent may be configured for disposal within endplate member 30 and/or permanent implant member 34. The at least one agent can include bone growth promoting material, such as, for example, a bone graft. The bone graft can be a particulate material, which may include an osteoconductive material such as hydroxyapatite and/or an osteoinductive agent such as a bone morphogenic protein (BMP) to enhance bony fixation of endplate member 30 with the endplates of adjacent vertebrae.

It is contemplated that the bone graft may include therapeutic polynucleotides or polypeptides. It is further contemplated that the bone graft may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines.

It is envisioned that the agent may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, periicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An interbody implant system comprising:
at least one endplate member defining a longitudinal axis, the endplate member configured for engagement with a vertebral endplate and permanent implantation; and
a permanent implant member having a selected configuration and being disposed adjacent the at least one endplate member within the intervertebral space, wherein the at least one endplate member comprises a first member defining a first longitudinal axis and being engageable with a first vertebral endplate, and a second member defining a second longitudinal axis and engageable with a second opposing vertebral endplate.

2. The interbody implant system of claim 1, further comprising a trial member configured for disposal adjacent the at least one endplate member within an intervertebral space wherein the permanent implant member has a configuration and dimension corresponding to the trial member.

3. The interbody implant system of claim 1, wherein the first member has a first coupling part and the second member has a second coupling part, the coupling parts comprising a cavity and a protrusion disposed in the cavity.

4. The interbody implant system of claim 3, wherein the protrusion is fixed with the cavity in a configuration to align the first and second members with the intervertebral space.

5. The interbody implant system of claim 2, wherein the at least one endplate member is configured to prevent undesirable engagement of the trial member with the vertebral endplate.

6. The interbody implant system of claim 1, wherein the at least one endplate member comprises an instrument extending therefrom for manipulation of the at least one endplate member in the intervertebral space.

7. The interbody implant system of claim 6, wherein the instrument is disposed at an angular orientation relative to the longitudinal axis.

8. The interbody implant system of claim 6, wherein a U-joint connects the instrument to the at least one endplate member.

9. The interbody implant system of claim 6, wherein the instrument includes a catch for fixed engagement with a retractor.

10. The interbody implant system of claim 2, wherein the trial member is configured to determine intervertebral space height and/or lordotic angle.

11. The interbody implant system of claim 1, wherein the at least one endplate member is anatomically configured for alignment with the vertebral endplate.

12. The interbody implant system of claim 2, wherein the at least one endplate member has a first surface and second surface oriented in an opposing direction, the second surface defining a first coupling part, and the trial member or the permanent implant member defining a second coupling part, the coupling parts comprise a cavity and a protrusion disposed in the cavity.

13. The interbody implant system of claim 12, wherein the cavity includes a keyway and the protrusion includes a rail.

14. The interbody implant system of claim 12, wherein the cavity includes a stop configured to engage the protrusion to prevent movement of the trial member or the permanent implant member.

15. The spinal implant of claim 1, further comprising a backout prevention member connected to the at least one endplate member and configured for engagement with the permanent implant member.

16. An interbody implant system comprising:
a first member configured for alignment with a first vertebral endplate surface and permanent implantation therewith, the first member defining a longitudinal axis, a first surface engageable with the first vertebral endplate surface and a second surface oriented in an opposing direction, the second surface defining a first keyway;
a second member configured for alignment with a second vertebral endplate surface and permanent implantation therewith, the second member defining a longitudinal axis, a first surface engageable with the second vertebral endplate surface and a second surface oriented in an opposing direction, the second surface of the second member defining a second keyway;
a trial member configured for removable disposal with the first and second members between the first and second vertebral endplate surfaces, the trial member comprising a first rail disposable in the first keyway and second rail disposable in the second keyway; and
a permanent implant member having a configuration and dimension corresponding to the trial member and being disposed with the first and second members between the first and second vertebral endplate surfaces.

17. A method for treating a spine, the method comprising the steps of:
providing at least one endplate member;
delivering the at least one endplate member into an intervertebral space and into alignment with a vertebral endplate for permanent implantation;
providing at least one trial member;
delivering the at least one trial member adjacent the at least one endplate member within the intervertebral space;
selecting one of the at least one trial member to determine a configuration and dimension of the intervertebral space;
providing a permanent implant member based on the selected trial member; and
delivering the permanent implant member adjacent the at least one endplate member within the intervertebral space for permanent implantation.

18. The method for treating a spine of claim 17, wherein the step of delivering the at least one endplate member includes an instrument connected to the at least one endplate member at an angular orientation relative to a longitudinal axis of the at least one endplate member, and further comprising the step of manipulating the permanent implant member with the instrument.

19. The method for treating a spine of claim 17, further comprising the step of disposing a backout prevention member with the at least one endplate member to prevent movement of the permanent implant member.

20. The method for treating a spine of claim 17, wherein the at least one endplate member includes a first member and a second member, and further comprising the step of delivering the first member into alignment with a first vertebral endplate surface for permanent implantation, and delivering the second member into alignment with a second vertebral endplate surface for permanent implantation.
